(19) United States
(12) Patent Application Publication
(10) Pub. No.: US 2026/0132105 A1
(43) Pub. Date: May 14, 2026

(54) CLETHODIM AMINE SALT, PREPARATION METHOD AND USE THEREOF

(71) Applicant: YIFAN BIOTECHNOLOGY GROUP CO., LTD., Wenzhou (CN)

(72) Inventors: Yuyao ZENG, Wenzhou (CN); Aie WU, Wenzhou (CN); Yifan NIU, Wenzhou (CN); Yuyan FAN, Wenzhou (CN); Shihua HUANG, Wenzhou (CN); Weiwei ZHOU, Wenzhou (CN); Deqiang YE, Wenzhou (CN)

(21) Appl. No.: 19/440,745

(22) Filed: Jan. 6, 2026

Related U.S. Application Data
(63) Continuation of application No. PCT/CN2024/131990, filed on Nov. 14, 2024.

(30) Foreign Application Priority Data
Oct. 28, 2024 (CN) 202411509737.6

(51) Int. Cl. C07C 323/47; A01N 35/10; A01P 13/02

(52) U.S. Cl. CPC C07C 323/47; A01N 35/10; A01P 13/02; C07B 2200/13

(57) ABSTRACT

The present application discloses a clethodim amine salt, a preparation method and a use thereof. The clethodim amine salt is a clethodim tert-butylamine salt with a chemical structural formula as below:

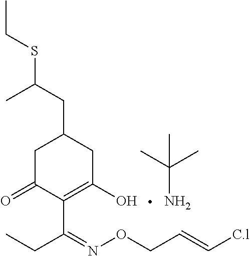

The clethodim tert-butylamine salt prepared in the present application has good stability, and at the same time maintains herbicidal activity not weaker than that of clethodim. Moreover, the stability and herbicidal activity of the clethodim tert-butylamine salt prepared in the present application are both superior to those of other clethodim amine salts reported in the literature. The clethodim tert-butylamine salt prepared in the present application has a high melting point and is a solid at room temperature. As a herbicide, it can be processed into more abundant formulations, extending from the current main formulation of clethodim emulsifiable concentrate (EC) to new formulations such as water-dispersible granules (WG) and wettable powders (WP), having good commercial prospects.

### CROSS-REFERENCE TO RELATED APPLICATIONS

The present application is a continuation application of PCT application No. PCT/CN2024/131990, filed on Nov. 14, 2024, which claims priority to Chinese patent application No. 202411509737.6, filed on Oct. 28, 2024. The entireties of PCT application No. PCT/CN2024/131990 and Chinese patent application No. 202411509737.6 are hereby incorporated by reference herein and made a part of this specification.

### TECHNICAL FIELD

The present application relates to the field of agricultural herbicides, and specifically to a clethodim amine salt, a preparation method and a use thereof.

### BACKGROUND ART

Clethodim, with a Chinese chemical name of 2-{1-[(3-chloro-2-allyl)oxy]iminopropyl}-5-[2-(ethylthio) propyl]-3-hydroxy-2-cyclohe xen-1-one, is a cyclohexanedione herbicide with excellent herbicidal activity. Currently, the commercially available industrial products of clethodim are in liquid form, with a component content of 86-93%. The molecular structure of clethodim contains multiple non-adjacent carbon-carbon double bonds, oxime groups and other functional groups, which lead to its poor stability and make the molecule susceptible to configuration inversion under the influence of external conditions and sensitive to ultraviolet light, heat, strong acids, strong bases, etc. Therefore, it is prone to decomposition during long-term transportation and storage, resulting in a decrease in the clethodim content in the product and a reduction in herbicidal efficacy. Once clethodim decomposes, the content of its active ingredients decreases, which severely affects the subsequent formulation processing and herbicidal efficacy. Therefore, it is very important to reduce the decomposition rate of clethodim technical materials and preparations during storage, improve their stability, and increase the content of active ingredients. Meanwhile, the commercially available clethodim preparations currently are all emulsifiable concentrate products (EC), such as 240EC and 360EC formulations, and there are no solid formulations, such as water-dispersible granules (WG) or wettable powder (WP), which also limits the application scenarios and commercial uses of clethodim.

Different clethodim amine salts will have different stabilities, different physical and chemical properties, and may also have different herbicidal activities and different herbicidal efficacies; different clethodim amine salts will bring different efficacies and effects, and the formulations and properties of economically important preparations may also be different.

Therefore, it is necessary to develop new derivatives of clethodim that exhibit one or more improved properties, such as improved storage stability, improved herbicidal activity and efficacy.

### SUMMARY

To solve the issues of poor stability and rapid weakening of herbicidal efficacy of clethodim, the present application provides a clethodim amine salt, a preparation method and a use thereof.

The above-mentioned first inventive purpose of the present application is achieved through the following technical solution:

A clethodim amine salt, which is a clethodim tert-butylamine salt, has a chemical structural formula as below:

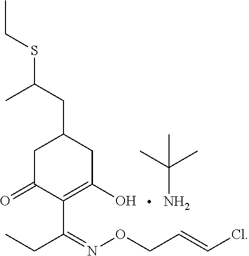

Optionally, the clethodim amine salt can be characterized through one or more ways as follows, including but not limited to high performance liquid chromatography (HPLC), nuclear magnetic resonance (HNMR), and a differential scanning calorimetry (DSC) curve showing an endothermic melting peak at 104° C. and a melting range of 93-107° C.

By adopting the above technical solution, the structure of the clethodim tert-butylamine salt in the present application is confirmed, including but not limited to nuclear magnetic resonance (HNMR), differential scanning calorimetry (DSC), etc. As shown in FIG. 1, the clethodim tert-butylamine salt of the present application is characterized by ¹H nuclear magnetic resonance (¹H NMR) spectrum. As shown in FIG. 2, the clethodim tert-butylamine salt of the present application is characterized by differential scanning calorimetry (DSC) spectrum.

The clethodim tert-butylamine salt prepared in the present application has good stability superior to that of clethodim and other clethodim amine salts, and at the same time maintains herbicidal activity not weaker than that of clethodim. The clethodim tert-butylamine salt has a lower degradation rate during the high-temperature heat storage than that of clethodim and other clethodim amine salts reported in the literature, so it is easier to store for a long time than clethodim.

The above-mentioned second inventive purpose of the present application is achieved through the following technical solution:

A method for preparing the above clethodim amine salt includes the following steps:
S1: dissolving clethodim in a solvent to obtain a mixture A;
S2: adding tert-butylamine into the mixture A to form a reaction system for reaction and precipitating the clethodim amine salt from the reaction system at an end of the reaction to obtain a precipitate;
S3: separating the precipitate to obtain the clethodim amine salt.

By adopting the above technical solution, the method for preparing the clethodim amine salt is simple, with mature process, good stability, high yield and low production cost. Moreover, compared with the currently commercialized clethodim technical materials, the content of the clethodim amine salt products can be significantly increased.

Optionally, a temperature of the reaction in the step S2 is −5° C. to 45° C.

By adopting the above technical solution, the reaction conditions for the method of the present application are mild, and the process is mature and safe.

Optionally, a feeding molar ratio of the clethodim to the tert-butylamine in the step S2 is (1:1.2)-(1:1).

By adopting the above technical solution, the yield and purity of the clethodim tert-butylamine salt products are both high.

Optionally, the clethodim amine salt obtained in S3 is further subjected to washing and purification.

By adopting the above technical solution, the purity of the clethodim tert-butylamine salt products is high.

The above-mentioned third inventive purpose of the present application is achieved through the following technical solution:

Use of the above clethodim amine salt in herbicide.

By adopting the above technical solution, the clethodim tert-butylamine salt exhibits good stability and herbicidal efficacy and therefore has the potential to be prepared into a commercial preparation with economic value, offering superior storage stability and excellent efficacy.

In summary, the present application at least has the following beneficial effects:
1. The present application discloses a clethodim tert-butylamine salt and characterizes it through various means. The long-term storage stability of the clethodim tert-butylamine salt of the present application is improved, which is better than that of not only clethodim but also other clethodim amine salts, while maintaining excellent herbicidal activity. Moreover, the stability and herbicidal activity of the clethodim tert-butylamine salt prepared in the present application are both superior to those of other clethodim amine salts reported in the literature.
2. The preparation method of the present application features mild reaction conditions, mature process, good stability, high yield and low production cost. Moreover, compared with the currently commercialized clethodim technical materials, the content of the clethodim amine salt products can be significantly increased.
3. The clethodim tert-butylamine salt of the present application has the potential to be prepared into a commercial preparation with economic value, offering superior storage stability and excellent efficacy. The clethodim tert-butylamine salt of the present application exhibits a lower degradation rate during the high-temperature heat storage process than clethodim and other clethodim amine salts reported in the literature, so it is easier to store for a long time than clethodim, which is conducive to the stability of commercial products during the shelf life. At the same time, it is beneficial to change the singularity of the current clethodim commercial formulations and expand to clethodim solid preparations, including new formulations such as water-dispersible granules (WG) or wettable powder (WP) with good commercial prospects.

### BRIEF DESCRIPTION OF THE DRAWINGS

Various features and aspects of the embodiments of the present application disclosed herein can be more clearly understood by referring to the accompanying drawings. The accompanying drawings are intended to illustrate and explain rather than limit the scope of the present application, in which:

FIG. 1 shows the 1H nuclear magnetic resonance (1H NMR) spectrum of the clethodim tert-butylamine salt.

FIG. 2 shows the differential scanning calorimetry (DSC) spectrum of the clethodim tert-butylamine salt.

### DETAILED DESCRIPTION

### Example 1

A clethodim amine salt is a clethodim tert-butylamine salt with a chemical structural formula as below:

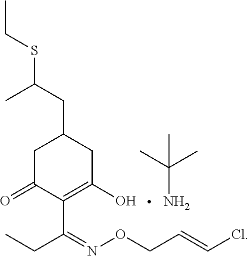

A method for preparing the clethodim tert-butylamine salt was as follows:
S1: 40.2 g clethodim technical materials (89.5 wt %, 0.10 mol) and 25 g solvent A were mixed uniformly with stirring at 16° C., then a mixing temperature was heated to 18° C., 7.39 g tert-butylamine liquid (99 wt %, 0.1 mol) was added dropwise at a constant rate for 5 minutes, and a resulting mixture was stirred at 18° C. for 1.0 hour, and then 120.0 g solvent B was added to obtain a mixture A;
S2: the mixture A was continually stirred at 15° C. for 5.0 hours for reaction to obtain a reaction material;
S3: the reaction material was filtered to obtain a solid;
S4: the solid was washed with solvent B in an amount of 5 times by mass, followed by vacuum dried at 20° C. to obtain a product, namely the clethodim tert-butylamine salt.

The solvent A was toluene, and the solvent B was cyclohexane.

### Examples 2 to 7

A clethodim tert-butylamine salt is different from Example 1 in that the process parameters of the preparation method are different, and the specific differences are shown in Table 1.

TABLE 1
Parameters of Preparation Methods of Examples 1 to 7

| Process | Example | Example | Example | Example | Example | Example | Example |
|---|---|---|---|---|---|---|---|
| parameters | 1 | 2 | 3 | 4 | 5 | 6 | 7 |
| Mixing | 18 | 16 | 16 | 16 | 16 | 16 | 16 |
| temperature/° C. | | | | | | | |
| Amount of | 40.2 | 40.2 | 40.2 | 40.2 | 40.2 | 40.2 | 40.2 |
| clethodim | | | | | | | |
| technical | | | | | | | |
| materials/g | | | | | | | |
| Solvent A | Toluene | Toluene | Toluene | Toluene | Toluene | Toluene | 1,2-dichloroethane |
| Amount of | 25 | 25 | 25 | 25 | 25 | 25 | 25 |
| solvent A/g | | | | | | | |
| Amount of | 7.39 | 8.13 | 8.13 | 8.13 | 8.86 | 11.06 | 8.13 |
| tert-butylamine | | | | | | | |
| liquid/g | | | | | | | |
| Molar ratio of | 1:1 | 1:1.1 | 1:1.1 | 1:1.1 | 1:1.2 | 1:1.5 | 1:1.1 |
| clethodim to | | | | | | | |
| tert-butylamine | | | | | | | |
| Solvent B | Cyclohexane | Cyclohexane | Cyclohexane | Cyclohexane | Cyclohexane | Cyclohexane | Cyclohexane |
| Amount of | 120 | 120 | 120 | 90 | 120 | 120 | 120 |
| solvent B/g | | | | | | | |
| Reaction | 15 | 15 | −5 | 45 | 15 | 15 | 15 |
| temperature/° C. | | | | | | | |
| Reaction time | 5 | 5 | 9 | 3 | 5 | 5 | 5 |
| with stirring/h | | | | | | | |

The products obtained in Examples 1 to 7 were subjected to high performance liquid chromatography (HPLC) for content detection and yield calculation, with the results listed in Table 2.

TABLE 2
Purity and Yield of Products of Examples 1 to 7

| Process | Example | Example | Example | Example | Example | Example | Example |
|---|---|---|---|---|---|---|---|
| parameters | 1 | 2 | 3 | 4 | 5 | 6 | 7 |
| Content/% | 94.5 | 99.3 | 93.8 | 91.3 | 96.5 | 91.6 | 89.5 |
| Yield/% | 91.6 | 95.7 | 94.6 | 90.4 | 92.8 | 90.7 | 85.2 |

It can be known from Table 1 and Table 2 that when clethodim reacts with tert-butylamine to obtain the target product of the present application, to ensure the effective utilization of clethodim, the yield can be improved by appropriately increasing the amount of tert-butylamine. It can be known from the comparison between Examples 1 to 2 and Examples 5 to 6 that too much excessive tert-butylamine has a negative impact on the yield and content of the product. Considering both the production cost and the final output index, it is preferable to control the molar ratio of clethodim to tert-butylamine at (1:1) to (1:1.2) in the present application.

It can be known from Examples 1 to 4 that when obtaining the clethodim tert-butylamine salt of the present application by the preparation method of the present application, a high purity of target product can be obtained in high yield by stably controlling the reaction temperature within −5 to 45° C. while appropriately adjusting the reaction time. At lower temperatures, the required reaction time is longer. This preparation method features low production cost, mature and simple preparation method, good stability, and high yield.

It can be known from Example 2 and Example 7 that the liquid-phase environment used in the reaction of clethodim tert-butylamine salt in the present application can be one of chloroalkane-based and benzene-based solvents.

The product obtained from Example 2 with the highest product purity was selected as the test sample for the stability test, with a melting point of 92 to 94° C.

### Comparative Example 1

A clethodim amine salt is different from Example 2 in that the amine used in the step S2 was sec-butylamine, the molar ratio of clethodim to sec-butylamine was 1:1.1, and the clethodim amine salt obtained is specifically shown in Table 3.

### Comparative Example 2

A clethodim amine salt is different from Example 2 in that the amine used in the step S2 was isobutylamine, the molar ratio of clethodim to isobutylamine was 1:1.1, and the clethodim amine salt obtained is specifically shown in Table 3.

### Comparative Examples 3 to 7

A clethodim amine salt is different from Example 2 in that the amine used in the step S2 was different, the molar ratio of clethodim to the amine is 1:1.1, and the clethodim amine salt obtained is specifically shown in Table 3.

The clethodim amine salts obtained in Comparative Examples 1 to 7 were subjected to content and melting point detection as well as yield calculation, with the results recorded in Table 3.

Twelve samples were taken from the same test substance for the melting point measurement. The maximum deviation among the measured results was within 5° C., and the distribution range of the melting point results was recorded. If the maximum deviation was greater than 5° C., the test substance was re-prepared, and samples were re-collected for testing.

TABLE 3
Information, Product Content, and Yield of Amines Used in Comparative Examples 1 to 7

| | | Content of | Melting point | Yield of |
|---|---|---|---|---|
| | Name of | clethodim amine | of the product | clethodim |
| Comparative Example No. | amine uesd | salt (%) | (° C.) | amine salt (%) |
| Comparative Example 1 | Sec-butylamine | 98.69 | 78-83 | 90 |
| Comparative Example 2 | Isobutylamine | 97.05 | 75-78 | 92 |
| Comparative Example 3 | Methylamine | 95.05 | 69-72 | 90 |
| Comparative Example 4 | Ethylamine | 99.60 | 82-87 | 91 |
| Comparative Example 5 | n-propylamine | 96.27 | 72-75 | 95 |
| Comparative Example 6 | Isopropylamine | 93.91 | 78-82 | 95 |
| Comparative Example 7 | n-butylamine | 90.52 | /* | 86 |

*In the melting point test of the clethodim amine salt obtained in Comparative Example 7, the melting point fluctuation range exceeded 10° C, and multiple re-samplings and measurements still failed to yield test results with a fluctuation range within 5° C. It was found after analysis that due to the low content of the clethodim amine salt, the decomposition of clethodim or other by-products during heating caused the decomposition of the clethodim amine salt.

The samples obtained from Example 2 and Comparative Examples 1 to 7 were tested for the stability by accelerated storage testing.

According to the regulations in CIPAC MT 46.4 Accelerated Storage procedure of the international method for the physical and chemical determination of pesticide technical materials and preparations-CIPAC method, the shelf life of pesticide technical materials and preparations under standardized operations is at least 2 years, and the shelf life of pesticide technical materials and preparations under standardized operations can be tested by accelerated storage testing. According to the regulations in CIPAC MT 46.4 Accelerated Storage procedure, the following 6 groups of combinations of storage temperature and storage period were used for accelerated storage testing, and the test results of the residual active ingredients in the samples can be regarded as predictive values for 2-year storage:
(1) 54 age:stofor 14 days;
(2) 50;ays;torage tempera
(3) 45;ays;torage tempera
(4) 40;ays;torage tempera
(5) 35±2° C., for 12 weeks;
(6) 30±2° C., for 12 weeks;

That is, storing at 54 eks;ure and storage period were used for accelerated storage testing, and the Among the combinations of storage temperature and storage period, the storage temperature has a predominant influence on the accelerated storage testing of pesticide technical materials and preparations, determines the cycle of accelerated storage testing, and reflects the upper limit of the extreme conditions tolerated by the pesticide technical materials and preparations during storage.

In the present application, orage temperature and storage period, the storage temperature has a predominant influence on the accelerated storage testing of pesticide technical materials and preparations, determines the cl materials and preparations-CIPAC method were cited herein. The rate-of-change curves of the samples are not identical under different parameters, and therefore there is no feasibility of conversion. For example, the sample situation at the condition of 54±2° C. for 7 days is not the same as that at the condition of 35±2° C. for 6 weeks.

The specific testing method was as follows: The sample was sealed in an ampoule bottle and stored in an incubator at 54±2° C. for 14 days. Then, the high-performance liquid chromatography analysis method was used to analyze and detect the purity change of the active ingredient before and after heat storage, and a commercially purchased clethodim sample (with a mass content of 89.50%) was used as the control example.

The test results are shown in Table 4.

TABLE 4
Stability Test Results of Example 2 and Comparative Examples 1 to 7

| | | Sample Content (%) and Decomposition Rate (%) | | | |
|---|---|---|---|---|---|
| | | after Different Heat Storage Periods | | | |
| | | | Heat | Heat | Heat |
| | | Initial | storage for | storage for | storage for |
| Samples for Heat Storage | | content | 3 days | 7 days | 14 days |
| Example 2 (clethodim | Sample content (%) | 99.3 | 97.81 | 91.43 | 88 |
| tert-butylamine salt) | Decomposition rate (%) | / | 1.50 | 7.93 | 11.38 |
| Comparative Example 1 | Sample content (%) | 98.69 | 84.67 | 49.29 | 1.9 |
| (clethodim | Decomposition rate (%) | / | 14.21 | 50.06 | 98.07 |
| sec-butylamine salt) | | | | | |
| Comparative Example 2 | Sample content (%) | 97.05 | 22.26 | 6.77 | 6.35 |
| (clethodim | Decomposition rate (%) | / | 77.06 | 93.02 | 93.46 |
| tert-isobutylamine salt) | | | | | |
| Comparative Example 3 | Sample content (%) | 95.05 | 26.47 | 7.17 | 1.58 |
| (clethodim methylamine | Decomposition rate (%) | / | 72.15 | 92.46 | 98.34 |
| salt) | | | | | |
| Comparative Example 4 | Sample content (%) | 99.6 | 41.23 | 13.91 | 1.94 |
| (clethodim ethylamine | Decomposition rate (%) | / | 58.60 | 86.03 | 98.05 |
| salt) | | | | | |
| Comparative Example 5 | Sample content (%) | 96.27 | 29.53 | 9.86 | 1.13 |
| (clethodim n-propylamine | Decomposition rate (%) | / | 69.33 | 89.76 | 98.83 |
| salt) | | | | | |
| Comparative Example 6 | Sample content (%) | 93.91 | 49.53 | 24.43 | 4.49 |
| (clethodim | Decomposition rate (%) | / | 47.26 | 73.99 | 95.22 |
| iso-propylamine salt) | | | | | |
| Comparative Example 7 | Sample content (%) | 90.52 | 21.39 | 4.87 | 0.73 |
| (clethodim n-butylamine | Decomposition rate (%) | / | 76.37 | 94.62 | 99.19 |
| salt) | | | | | |
| Control Example | Sample content (%) | 89.5 | 85.03 | 76.86 | 49.2 |
| (clethodim) | Decomposition rate (%) | / | 4.99 | 14.12 | 45.03 |

Sample content is the content of corresponding active ingredients (clethodim amine salt or clethodim) in the sample after heat storage. Decomposition rate is calculated according to the formula below: Decomposition rate (%)=(Initial content−Sample content after heat storage)/Initial content×100%.

During the research process of the present application, it was found that when stored at 54 below: Decomposition rate (%)=(Initial content−Sample content after heat storage)/Initial content×100%.ly purchasvely compare Example 2 with Comparative Examples 1 to 7, in addition to the 14 days of storage at 54±2° C. required by the CIPAC MT 46.4 Accelerated Storage procedure, this application independently conducted additional storage tests on the same samples at 54±2° C. for 3 days and 7 days to better monitor the decomposition during storage and reflect the stability performance of each sample.

It can be known from Table 4 that after accelerated storage at 54 nd that when stored at 54 below: Decomposition rate in the samples of Comparative Examples 1 to 7 and the control example, while the decomposition in Example 2 was gentle and the final decomposition rate after 14 days was much lower than that of Examples 1-7. Therefore, the stability of clethodim amine in the present application is better than that of clethodim and other clethodim amine salts, and it is reasonably inferred that the prepared technical materials or preparations are not easily decomposed during long-term storage.

In addition, samples were obtained from Example 2 (clethodim tert-butylamine salt), Comparative Example 1 (clethodim tertiary and/or secondary amine salt), and Comparative Example 2 (clethodim tert-isoamine salt) for herbicidal activity testing.

Herbicidal activity testing: The testing was carried out according to the method of Herbicides-Part 4: Activity Determination Test-Stem and Leaf Spraying Method (NY/T 1155.4-2006) in the Guidelines for Pesticide Bioassay in the Laboratory. The test targets were barnyard grass, Polypogon fugax, and goose grass, and a commercially purchased clethodim sample (with a content of 89.50%) was used as the control example. The test results showed the fresh weight control effects against weeds after 21 days under different dosages.

The herbicidal activity test results are shown in Table 5.

TABLE 5
Herbicidal activity test results of Example 2 and Comparative Examples 1 to 2*

| Source and | Dosage of 6.25 g a.i./hm² | | | Dosage of 12.5 g a.i./hm² | | |
|---|---|---|---|---|---|---|
| Name of | Barnyard | Polypogon | Goose | Barnyard | | Goose |
| Herbicidal Test | grass | fugax | grass | grass | Polypogon | grass |
| Samples | (%) | (%) | (%) | (%) | fugax (%) | (%) |
| Example 2 (clethodim | 88.031 | 89.69 | 66.25 | 99.33 | 98.87 | 95.59 |
| tert-butylamine salt) | | | | | | |
| Control Example | 97.52 | 97.83 | 67.34 | 98.71 | 100 | 89.28 |
| (clethodim) | | | | | | |
| Comparative | 68.27 | 86.27 | 59.86 | 87.32 | 84.47 | 73.01 |
| Example 1 (clethodim | | | | | | |
| sec-butylamine salt) | | | | | | |
| Comparative | 50.55 | 72.99 | 47.17 | 82.59 | 86.56 | 62.22 |
| Example 2 (clethodim | | | | | | |
| iso-butylamine salt) | | | | | | |

*Note:
the table shows the test results of the fresh weight control effect of the drug against different weeds after 21 days under two dosages.

Analysis of test results: when the dosage was 6.25 g a.i./hm2, 21 days after application, the herbicidal activities of different agents against barnyard grass from low to high were as follows: Comparative Example 2<Comparative Example 1<Example 2<Control Example; the herbicidal activities against Polypogon fugax from low to high were as follows: Comparative Example 2<Comparative Example 1<Example 2<Control Example; the herbicidal activities against goose grass from low to high were as follows: Comparative Example 2<Comparative Example 1<Example 2<Control Example; when the dosage was 12.5 g a.i./hm2, 21 days after application, the herbicidal activities of different agents against barnyard grass from low to high were as follows: Comparative Example 2<Comparative Example 1<Control Example<Example 2; the herbicidal activities against Polypogon fugax from low to high were as follows: Comparative Example 1<Comparative Example 2<Example 2<Control Example; the herbicidal activities against goose grass from low to high were as follows: Comparative Example 2<Comparative Example 1<Control Example<Example 2.

Based on the results in Table 5, it can be seen that the fresh weight control effect of Example 2 is similar to that of the Control Example, with good herbicidal activity and no obvious attenuation. Even when the dosage was 12.5 g a.i./hm2, the herbicidal activities against barnyard grass and goose grass were slightly better than those of the Control Example.

Comparative Examples 1 to 2 showed a significant decrease in herbicidal activity at different dosages compared with the Control Example.

It can be seen from the above data that the physical and chemical properties of different clethodim amine salts are different, resulting in obvious differences in their stability and herbicidal activity. Even for the clethodim amine salts (clethodim tert-butylamine salt, clethodim sec-butylamine salt, clethodim isobutylamine salt) formed by the isomers of amine (tert-butylamine, sec-butylamine, isobutylamine) and clethodim, there are significant differences in storage stability and herbicidal activity. In addition, the process difficulty, product performance and economic cost of processing different clethodim amine salts into various formulations may also be different. The clethodim tert-butylamine salt disclosed in the present application is a compound with very excellent comprehensive performance and broad commercial prospects.

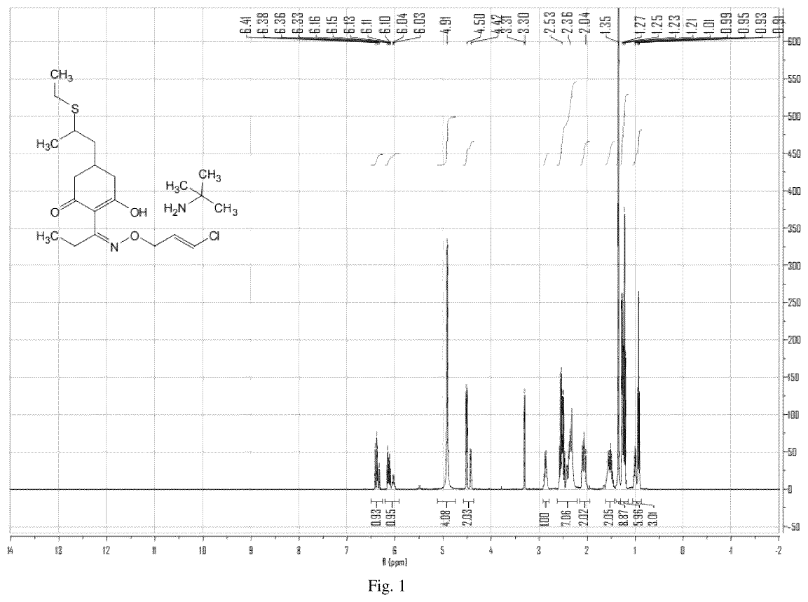

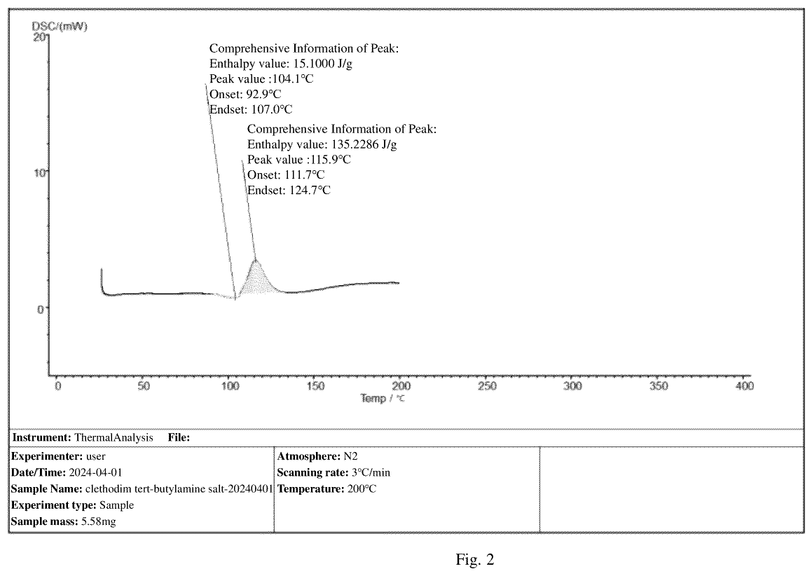

In addition, the sample of Example 2 was detected by 1H nuclear magnetic resonance spectrum and differential scanning calorimetry to further determine the structure of the sample of Example 2. The detection diagrams are shown in FIG. 1 and FIG. 2.

These specific examples are merely an explanation of the present application, rather than a limitation of the present application. After reading this specification, a person skilled in the art can make modifications to these examples without any creative contribution as desired, but these modifications are protected by the patent law as long as they are within the scope of the claims of the present application.

What is claimed is:

1. A clethodim amine salt, wherein the clethodim amine salt is a clethodim tert-butylamine salt with a chemical structural formula as below:

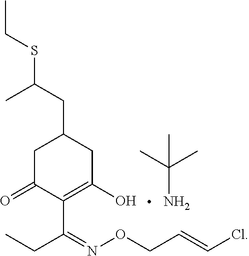

2. The clethodim amine salt according to claim 1, wherein the clethodim amine salt is characterized through one or more ways as follows: high performance liquid chromatography, nuclear magnetic resonance, and a differential scanning calorimetry curve showing an endothermic melting peak at 104° C. and a melting range of 93-107° C.

3. A method for preparing the clethodim amine salt of claim 1, comprising the following steps:
S1: dissolving clethodim in a solvent to obtain a mixture A;
S2: adding tert-butylamine into the mixture A to form a reaction system for reaction and precipitating the clethodim amine salt from the reaction system at an end of the reaction to obtain a precipitate;
S3: separating the precipitate to obtain the clethodim amine salt.

4. The method for preparing the clethodim amine salt according to claim 3, wherein a temperature of the reaction in the step S2 is −5° C. to 45° C.

5. The method for preparing the clethodim amine salt according to claim 3, wherein a feeding molar ratio of the clethodim to the tert-butylamine in the step S2 is (1:1)-(1:1.2).

6. The method for preparing the clethodim amine salt according to claim 3, wherein the clethodim amine salt obtained in the step S3 is further subjected to washing and purification.

7. Use of the clethodim amine salt of claim 1 in herbicide.
